(19) United States
(12) Patent Application Publication
(10) Pub. No.: US 2017/0164819 A1
(43) Pub. Date: Jun. 15, 2017

(54) Light Field Capsule Endoscope

(71) Applicant: Kevin Bai, atlanta, GA (US)

(72) Inventor: Kevin Bai, atlanta, GA (US)

(21) Appl. No.: 14/967,326

(22) Filed: Dec. 13, 2015

(51) Int. Cl. A61B 1/04; A61B 1/06; A61B 1/00; G02B 3/00; G02B 7/02

(52) U.S. Cl. CPC A61B 1/041; G02B 3/0006; G02B 7/021; A61B 1/00032; A61B 1/00016; A61B 1/0002; A61B 1/00006; A61B 1/0684

(57) ABSTRACT

A light field capsule endoscope with refocusable image function comprising of: A transparent body capsule with internal illumination sources; A lens at a fixed focal distance and its corresponding sensor system to capture images; A redundant light field lens and sensor system containing a microlens array with a plurality of microlenses to capture refocasable images; An onboard memory module; A wireless transmitter; An antennae.

### FIELD OF THE INVENTION

This invention relates to a light field capsule camera with applications in medical endoscopy

### BACKGROUND OF THE INVENTION

An encapsulated camera is a small digital device used for gastrointestinal (GI) endoscopy procedures for screening for diseases. In contrast to the gold standard metallic endoscope, the capsule is less invasive, with fewer risks for complication and reduced need for anesthetics during the procedure, which has contributed in its increasing popularity for diagnosis of GI diseases. As described by U.S. Pat. No. 5,604,531 to Iddan 1997, the device consists of an optical lens and sensor, along with a power source, and radio transmitter sandwiched inside an easily swallowed pill. The transmitter inside the pill sends captured image signals to a receiver outside of the body that is typically worn as a harness around the torso. The capsule might also contain a radio receiver for changing frequency of taking pictures or other adjustments. Other designs might also vary the power source to an internal or external inductor, or size of frequency used in transmission. Once swallowed, the capsule endoscope will take somewhere from eight to over ten hours, based on patient, to traverse the gastrointestinal tract, during which the patient will need to wear a wireless data receiver pack, before passing out of the body.

Because of size limitations, the main optical lens within the capsule camera operates with a fixed focal distance. Because objective distance for the lens is measured in terms centimeters in GI cavity, depth of field of a lens from scene is greatly depressed so a clear image is hard to obtain in many situations. Because of this extremely small size, the regular lens group or advanced focus control mechanism simply can not be applied in construction of capsule camera.

For a typical GI endoscopy process, thousands of images are generated. However, typically only a dozen of them will of necessary clarity at the correct locations to be useful for any diagnosis of diseases. If one of them is out of focus, there is no way to make up the loss other than to repeat with a brand new endoscopy process. With light field technology applied to the designs of new digital camera, it is becoming possible to take a photo first then generate a clear image later with computational photography programs.

In regular digital cameras, only intensity of incoming light is recorded on the image sensor. Installing a microlens array in between the main optical lens and its corresponding image sensor, turns regular camera into a light field camera, as described by U.S. Pat. No. 8,724,014 to Ng 2014. This is because the incoming light rays is separated by the microlens array then refocused into the sensor plane, the angular information is recorded together with the intensity information (FIG. 1). A clear image out of a blurred one can be generated later by a computer. However, the image obtained through a light field sensor have a substantially lower spatial resolution compared to the native spatial resolution of the same image sensor as described in U.S. Pat. No. 8,593,564B2 to Border 2013. Furthermore, a lot more man power is required to process every image generated from the light field endoscopy. Combination of a redundant light field image generating system with a regular image generation system in a single capsule camera makes it possible to obtain both regular clear images together with refocusable images. The blur images may be replaced by corresponding refocusable images with minimum amount of work.

### SUMMARY OF THE INVENTION

Capsule camera system with image refocusable function comprising: A transparent capsule body with internal illumination sources; An imaging lens and its sensor system to capture digital image; A redundant imaging lens and sensor system which contains microlens array with a plurality of microlenses, where the microlens array located between the redundant imaging lens and its sensor to capture refocusable digital image. Introduction of microlens array produces a light field capsule camera. This optical arrangement makes it possible for the image sensor to store additional light information generated from the entire three dimensional light field arising from incoming rays in order to create a series of images encompassing a spectrum of focal lengths which can be used to generate clear images out of blurred ones during post-procedural viewing.

In a typical embodiment of this invention, the redundant image information is either transferred wirelessly to outside receiver, or temporarily stored on board the capsule, while regular image is broadcasting wirelessly to the receiver outside patient body.

This invention has the advantage that blurred image can be peer-investigated through the redundant image collection and replaced with a clear focused image. It has the additional advantage that only a few critical image need this additional catch up review work to make sure a successful GI endoscopy with only one time camera pass.

### BRIEF DESCRIPTION OF THE DRAWINGS

FIG. 1 is a light field sensor principle

FIG. 2 is a regular capsule camera

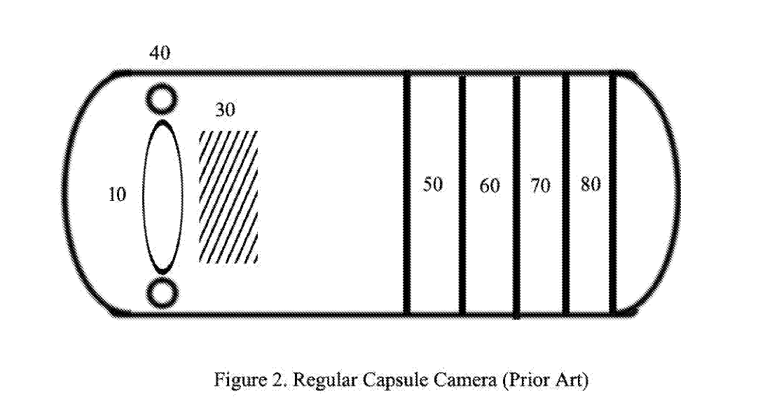

FIG. 3 is a light field capsule camera

FIG. 4 is a schematic drawing of an embodiment of the camera with a redundant image system with lens located on the opposite side of the camera

FIG. 5 is a schematic drawing of an embodiment of the camera with a redundant image system with lens using one sensor

### DETAILED DESCRIPTION OF THE INVENTION

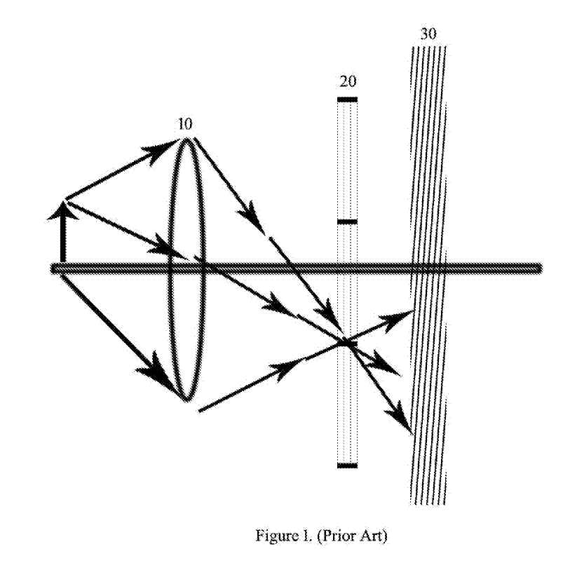

Regular capsule camera comprises major components of an optical lens, image sensor, internal light source, battery, radio transmitter or on board memory storage module as illustrated in FIG. 1.

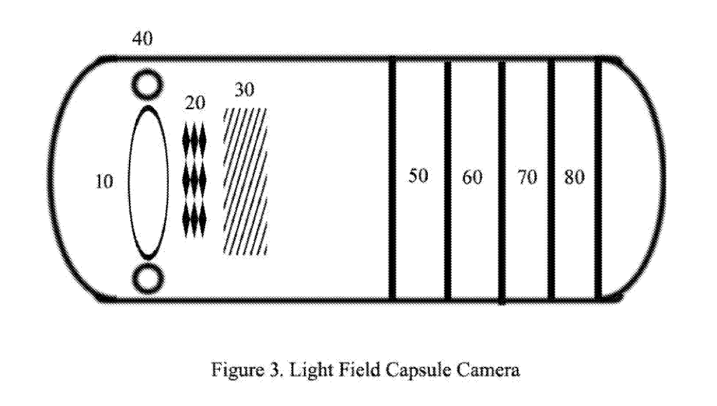

Placement of a microlens array in between major lens and the corresponding image sensor, a regular capsule camera effectively turns into a light field capsule camera, with function of image refocusing (FIG. 3). The refocusing process may be performed post endoscopy, with aid of computer software. The distance of the microlens array to the major lens (10) and the image sensor (20) is predetermined and fixed by a lens holder. In one embodiment, the microlens array is located on the focal plane of the major lens, while the image sensor is located on the microlens focal plane. Image information then broadcast wirelessly through the on board transmitter (30) or stored in a memory chip on board (40).

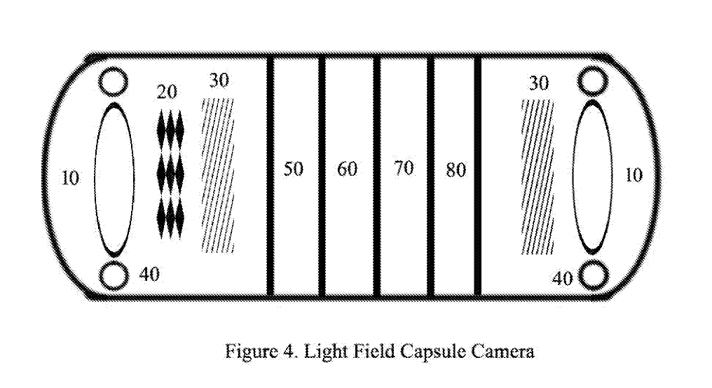

FIG. 4 illustrate the structure of an embodiment of a light field capsule camera with a redundant image system. The light field lens and sensor is placed on one end of the capsule, the redundant regular lens and sensor is placed on the opposite end of the capsule. Regular image signal is broadcasting through the transmitter, while light field image signal is stored on board in memory chips or transferred wirelessly to an outside receiver. Because the two redundant lens has a fixed distance from each other, any blurred regular image may be recovered from the light field image set which located with a similar time or location mark. This way, only limited number of light field image need to be refocused and processed to replace the regular blurred image, resulting in limited time commitment from the medical personnel.

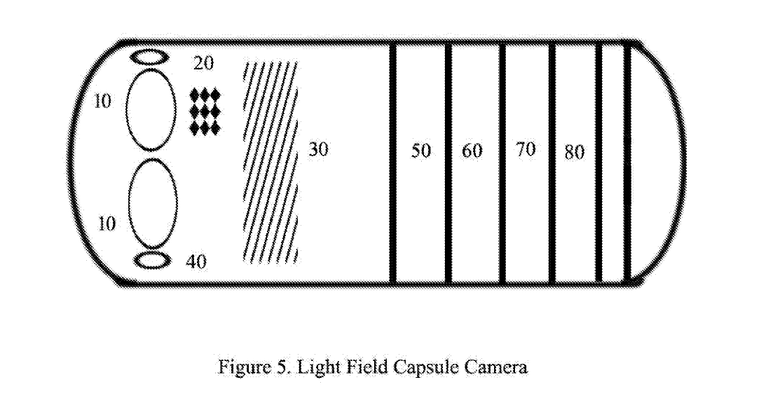

FIG. 5 illustrate the structure of a variation of embodiment of a light field capsule camera with a redundant image system. In this design, a regular lens (10) and a light field microlens array (20) generate two set of images on the same image sensor, but on a different region. As described by U.S. Pat. No. 8,724,868B2 to Wang 2014, the sub set of the different image may be individually arranged and processed. Both regular image signal and light field image signal are broadcasting through the transmitter to an outside receiver for further process.

1. A light field capsule endoscope consisting of:
a. A non-digestible, transparent outer protective shell capable of passing safely through a human gastrointestinal tract through peristaltic muscle contraction;
b. Plural of optical lens to generate image on plural of image sensors
c. An array of microlenses placed between an optical lens and its image sensor at a predetermined distance;
d. plural of lens holders that secures both the optical and microarray lenses;
e. A plurality of illumination sources such as LEDs;
f. A power source, such as batteries;
g. A control module;
h. plural memory module
i. A wireless communication module;
j. An antenna;

2. The light field capsule camera of claim 1 wherein the microlens array of plurality of microlens is positioned between an optical lens and its sensor;

3. The light field capsule camera of claim 1 wherein image generated from microlens array and its sensor is redundant to image from another lens and its sensor.
